(19) United States
(12) Patent Application Publication
(10) Pub. No.: US 2005/0273126 A1
(43) Pub. Date: Dec. 8, 2005

(54) Color treated condition-indicating ultrasonic surgical device and method

(75) Inventor: Jean M. Beaupre, Cincinnati, OH (US)

Correspondence Address: JAMES C. EAVES JR.;GREENEBAUM DOLL & MCDONALD PLLC, 3500 NATIONAL CITY TOWER, 101 SOUTH FIFTH STREET, LOUISVILLE, KY, 40202, US

(73) Assignee: Crescendo Technologies, Inc.

(21) Appl. No.: 11/145,640

(22) Filed: Jun. 6, 2005

Related U.S. Application Data
(60) Provisional application No. 60/577,545, filed on Jun. 7, 2004.

(51) Int. Cl. A61B 17/32

(52) U.S. Cl. 606/169

(57) ABSTRACT

Disclosed is a condition-indicating ultrasonic surgical device and method of making the same in which an ultrasonic surgical instrument (10) has at least a portion of which has an outer surface which includes a color layer formed by anodizing or chemical processing. The color layer has a predetermined thickness wherein the thickness, and thereby the color, of the layer is affected by damage to the instrument to provide an indication that would otherwise be imperceptible by visual inspection.

### RELATED APPLICATION

This application claims priority based on a U.S. provisional application titled Colored Ultrasonic Device and Method, Ser. No. 60/577,545, filed Jun. 7, 2004.

### TECHNICAL FIELD

The present invention relates, in general, to ultrasonic surgical devices and, more particularly, to a color treated ultrasonic surgical devices capable of visually indicating wear and damage.

### BACKGROUND

During surgery, a surgeon must both incise living tissue and control the resulting bleeding. This is traditionally done by cutting with a scalpel and tying off larger vessels with suture. This still leaves numerous smaller vessels to bleed. A very old technique of applying heat to wounds, or cauterizing them, is still used to stop bleeding, or coagulate. A significant advance was the introduction of instruments which pass a current through the tissue to heat and cauterize the tissue as it is cut. The electric current itself may be used to cut tissue when properly controlled. However, electrocautery, with enough intensity to cut, tends to desiccate and char tissue.

More recently, ultrasonic surgical devices have been introduced which permit effective cutting with reduced desiccation and charring. Initial work with these devices (Vang U.S. Pat. No. 2,714,890, Shafer U.S. Pat. No. 2,845,072, Balamuth U.S. Pat. No. 3,086,288) focused on improving cutting effects. In short, a vibrating cutting instrument would have advantages when incising tissue. Later work (Balamuth U.S. Pat. No. 3,636,943) noted that the heating action of a vibrating blade can also be used to control bleeding while cutting.

In ultrasonic surgical devices currently marketed, a power source, or generator, supplies a high frequency AC electrical signal to a hand held transducer. This transducer converts the electrical signal to longitudinal motion, a standing wave, using piezoceramic, magnetostrictive, or similar means. The transducer may mechanically amplify this motion using a horn or horns for delivery to an end effector. The transducer and end effector are composed of an integer number of half-wave wave guides designed to vibrate in a standing wave at the desired frequency. The end effector further amplifies the motion of the transducer, if necessary, to a useful level and transmits it to the functional portion of the device, which is shaped to perform a useful function. It is this end effector with its functional portion which, by action of its motion, cuts, and cauterizes. Devices using this effect are available from Ethicon Endo-Surgery, Cincinnati, Ohio; United States Surgical Corporation, Norwalk, Conn.; and Olympus Medical, Melville, N.Y.

The field of ultrasonic surgical devices has experienced a rapid growth due to the effectiveness of such devices in an increasing number of surgical procedures. However, unlike traditional scalpels, ultrasonic devices are subjected to considerable stress during use due to the high-frequency motion of the blades. This stress typically limits the useful life of such devices, where extended use may result in blade cracking and/or decreased blade functionality. Additionally, contact with other instruments and prolonged tissue contact may also accelerate the deterioration of the ultrasonic tool's usefulness. Scratches, mars, and stress fractures that indicate the initial stages of blade deterioration are often difficult to perceive. This difficulty is compounded when the device is used within the human body where the presence of blood and other bodily fluids makes detection even more difficult. Blade failure that results from failed detection of blade deterioration may result in unduly long procedures where blades must be changed or a decrease in tissue effect resulting from decreased blade sharpness. Additionally, where damage is difficult to detect in existing tools, ultrasonic blades may be disposed of before the end of their useful life to ensure that failures do not result during surgical procedures. Providing a means of early detection will maximize the useful life of such devices, thereby reducing costs and the potential damage resulting from the use of a defective blade. Single-use ultrasonic instruments represent a considerable percentage of the ultrasonic market. Although these devices are intended for single use only, ultrasonic instruments may be used inappropriately in multiple medical procedures.

It would therefore be advantageous to provide a device and method capable of providing early detection of damage to ultrasonic blades. It would be further advantageous to provide a device that maximizes the useful life of ultrasonic blades, thereby reducing costs and the likelihood of adverse effects due to blade failure. It would be further advantageous to provide a device and method that provided an early indication of early signs of blade failure in a consistent and uniform fashion. It would be further advantageous to provide a disposable blade that, upon use, indicated the blade should be disposed of and not used.

### SUMMARY OF THE INVENTION

The present invention provides a condition-indicating ultrasonic surgical device and method of making the same in which an ultrasonic surgical instrument has an outer surface at least a portion of which includes a color layer formed by anodizing or chemical processing. The color layer has a predetermined thickness wherein the thickness, and thereby the color, of the layer is affected by damage to the instrument to provide an indication that would otherwise be imperceptible by visual inspection.

### BRIEF DESCRIPTION OF THE DRAWING

FIG. 1 is a pictorial view of an ultrasonic instrument in accordance with the present invention; and

FIG. 2 is chart showing the resulting color and thickness resulting from anodizing voltages applied to titanium.

### DETAILED DESCRIPTION

Currently, there are no clearly visible indicators on previously used disposable blades to alert users and attending personnel that the ultrasonic instrument should be discarded. Such an indicator would help protect patient safety and would provide an additional assurance to manufacturers that disposable blades are not being abused.

The coloration of metals generally, by means such as anodizing, permanent inks, chemical vapor deposition, ion bombardment, chemical processing, chemical oxidation, and powder coating, is known in the art. The use of colorized materials in the medical field, however, has been used primarily to provide contrast with bodily tissue and to increase aesthetic appeal. For example, the surgical clips made my ETHICON ENDO-SURGERY, Cincinnati, Ohio, are an anodized blue to provide easy visibility and contrast with respect to bodily tissue. The color of anodizing metals, such as titanium, is produced by interference colors. In the anodizing process of titanium a thin layer of titanium oxide is produced. White light falling on the oxide is partially reflected and partially transmitted and refracted in the oxide film. The light that reaches the metal/oxide surface is mostly reflected back into the oxide. Several reflections may take place. A phase shift occurs during this process along with multiple reflections. The degree of absorption and number of reflections depends on the thickness of the film. The light that was initially reflected from the oxide surface interferes with the light that has traveled through the oxide and been reflected off the metal surface.

Depending on the thickness of the oxide, certain wavelengths (colors) will be in phase and enhanced while other wavelengths will be out of phase and damped. Hence, the observed color is mainly determined by the oxide thickness. Literature reports that oxide thickness is determined by the applied voltage with a growth constant of 2-3 nm per volt. At any given voltage the oxide film grows to a specific thickness and then stops when resistance increases to a point where no current is being passed. The phenomenon of voltage controlled oxide thickness indicates that the color is also voltage controlled. No such coloration has been applied to ultrasonic surgical devices that are not meant for temporary or permanent placement within the human body.

Presently, many ultrasonic surgical blades are produced having a faint yellow hue. This coloration is the result of a baking process that is performed on titanium used in such blades. A side effect of this process is a faint yellow tint. This tint, however, is ineffective in displaying to a user whether a device exhibits early signs of deterioration. The faint color provides little to no contrast, making the detection of scratches, mars, and stress fractures difficult. This difficulty is compounded during use within the human body due to the presence of blood and other bodily fluids. Additionally, the baking process does not produce a consistent surface coloration and is subject to multiple external factors such as temperature, time, and atmospheric conditions.

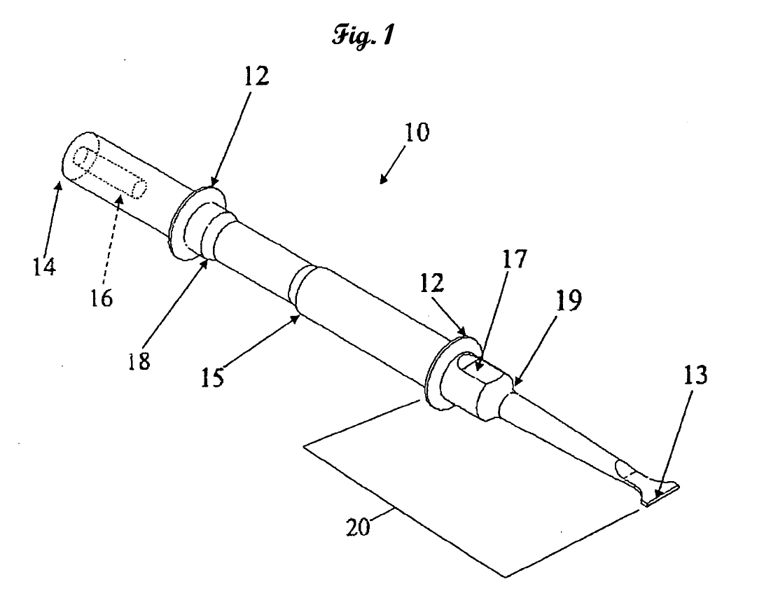

FIG. 1 illustrates one embodiment of an ultrasonic instrument 10 in accordance with the present invention. Embodiments of the present invention include the coloration of any suitable ultrasonic medical tool including, but not limited to, ultrasonic scalpels, ultrasonic hooks, ultrasonic balls, and ultrasonic shears. The term “ultrasonic instrument” shall herein be defined as any device vibrating at ultrasonic frequencies to perform medical procedures whether clinical or surgical. The disclosed embodiments are in no way intended to limit the scope of the appended claims. Ultrasonic instrument 10 includes silicon rings 12, a distal end 13, a proximal end 14, an antinode 15, a threaded connection 16, wrench flats 17, a proximal amplifier 18, a distal amplifier 19, and an end effector 20. The end effector 20 may be any suitable shape and/or configuration including a ball, hook, a spade, a crescent, a fork, a needle, a puncture device, or a shear.

Ultrasonic instrument 10 may, for example, be any suitable ultrasonic tool such as those disclosed in U.S. Pat. No. 2,990,616, U.S. Pat. No. 3,086,288, U.S. Pat. No. 3,636,943, U.S. Pat. No. 4,188,952, U.S. Pat. No. 5,263,957, U.S. Pat. No. 5,324,299, U.S. Pat. No. 5,669,922, U.S. Pat. No. 5,261,922, U.S. Pat. No. 5,322,055, U.S. Pat. No. 6,004,335, U.S. Pat. No. 6,309,400, U.S. Pat. No. 6,063,050, U.S. Pat. No. 5,318,570, and U.S. Pat. No. 5,324,297.

One embodiment of the present invention includes providing coloration to at least one portion of ultrasonic instrument 10 capable of indicating early signs of blade deterioration. “Coloration” is herein defined as having a wavelength within the light spectrum including, but not limited to, the visible light spectrum and wavelengths which are visible only with the assistance of external devices such as, for example, ultraviolet. In one embodiment of the present invention, ultrasonic instrument 10 is a titanium ultrasonic blade anodized, in its entirety, to the color dark blue using an applied voltage of about 20V. This will result in a titanium oxide film thickness of about 586 angstroms. Due to the phenomenon of voltage controlled oxide thickness, ultrasonic instrument 10 may be colored blue in a consistent and uniform fashion that makes any damage to the instrument visible. The coloration of an ultrasonic instrument, for example, dark blue, permits the early detection of scratches, mars and stress fractures that are not visible or are difficult to see in the absence of any coloration.

When damaged, in the anodized instrument embodiment of the present invention, the oxide film thickness on the instrument will decrease in thickness. Because this thickness corresponds to the visible color of the instrument, users will see a visible color change in the instrument when damage or scratching occurs. Because the relationship between color and oxide thickness is known, the visibility of a particular color will indicate to the user not only that the instrument has been damaged, but also the extent of damage. For example, an instrument anodized to a dark blue color may have an oxide thickness of 586 angstroms. If the instrument is damaged during use, reducing a portion of the instrument to an oxide thickness of 491 angstroms, the instrument will display a visible color of purple. Damage to the instrument that reduces the oxide thickness to about 362 angstroms will, for example, display a golden brown color. The uniformity in the application of the coloration makes such defects readily visible to the naked eye allowing the user to take action before the ultrasonic instrument fails.

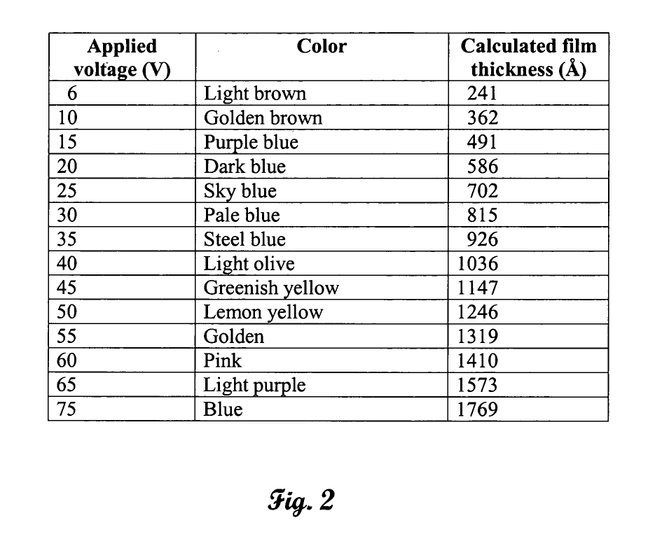

FIG. 2 discloses a chart displaying applied voltages to titanium and their corresponding thickness and visible color in accordance with multiple embodiments of the present invention. Disclosed by way of example only, the applied voltages, film thickness, and corresponding visible colors of FIG. 2 may be varied to provide the desired visible color to an instrument for a given application.

In accordance with one embodiment of the present invention, ultrasonic instrument 10 may be anodized with an applied voltage of about 6V in 165 g/L sulfuric acid at 23 degrees Celsius, resulting in a light brown color having calculated film thickness of about 241 angstroms, at an applied voltage of about 10V, resulting in a golden brown color having a calculated film thickness of about 362 angstroms, at an applied voltage of about 15V, resulting in a purple color having a calculated film thickness of about 491 angstroms, at an applied voltage of about 20V resulting in a dark blue color having a calculated film thickness of about 586 angstroms, at an applied voltage of about 25V, resulting in a sky blue color having a calculated film thickness of about 702 angstroms, at an applied voltage of about 30V, resulting in a pale blue color having a calculated film thickness of about 815 angstroms, at an applied voltage of about 35V, resulting in a steel blue color having a calculated film thickness of about 926 angstroms, at an applied voltage of about 40V resulting in a light olive color having a calculated film thickness of about 1036 angstroms, at an applied voltage of about 45V, resulting in a greenish yellow color having a calculated film thickness of about 1147 angstroms, at an applied voltage of about 50V, resulting in a lemon yellow color having a film thickness of about 1246 angstroms, at an applied voltage of about 55V, resulting in a golden color having a calculated film thickness of about 1319 angstroms, at an applied voltage of about 60V, resulting in a pink color having a calculated film thickness of about 1410 angstroms, at an applied voltage of 65V, resulting in a light purple having a calculated film thickness of about 1573 angstroms, or at an applied voltage of about 75V, resulting in a blue color having a calculated film thickness of about 1769 angstroms. The present invention includes providing an applied voltage of from about 3V to about 100V to titanium in order to modify the color of the material by providing an oxide film. A further embodiment of the present invention includes providing an applied voltage from about 20V to about 75V to titanium in order to modify the color of the material. Additional embodiments of the present invention include providing applied voltages from about 30V to about 60V, from about 35V to about 45V, from about 3V to about 35V, from about 35V to about 100V, or combinations thereof.

Embodiments of the present invention include constructing ultrasonic instrument 10 from any material suitable for use in ultrasonic applications such as, for example, titanium and its alloys, aluminum and its alloys, stainless steel and its alloys, ceramics, silicon, and combinations thereof. In addition to anodizing any metal, such as titanium, suitable for use in ultrasonics, additional embodiments of the present invention include providing coloration through chemical processing to ultrasonic instrument 10 by the application of a catalyst, energy or both including, but not limited to, permanent inks, chemical vapor deposition, powder coating, ion bombardment, chemical oxidation or combinations thereof.

One embodiment of a method in accordance with the present invention for providing coloration to an ultrasonic device includes providing an ultrasonic instrument and providing the ultrasonic instrument with coloration step. This step includes providing any ultrasonic instrument suitable for use in a medical capacity. Ultrasonic instruments in accordance with this method may be instruments suitable for clinical, laboratory, and/or surgical use. Another method includes providing coloration to all of a portion of the ultrasonic instrument of the previous method by, for example, anodizing, providing permanent inks, using chemical vapor deposition, providing a powder coating, chemical processing, chemical oxidation, chemical processing, and/or using ion bombardment.

A further embodiment of the present invention includes providing coloration to only a portion of ultrasonic instrument 10 such as, for example, to end effector 20. End effector 20 may be a distal portion of ultrasonic instrument 10 constructed from titanium that is anodized using an applied voltage of about 65V to provide the end effector with a light purple color. Providing coloration, such as light purple, to areas of high longitudinal stress, such as end effector 20, may permit early detection of blade deterioration. A further embodiment includes providing bands of coloration at antinodes and/or other areas prone to high stress and/or damage. Depending on the medical procedure, any suitable coloration may be provided to ultrasonic instrument 10 to provide optimal visibility of early signs of instrument failure. For example, a dark blue instrument may be used during procedures involving a great deal of blood of other bodily fluids.

A further embodiment of the present invention includes placing coloration on a portion of end effector 20 of ultrasonic instrument 10 to visually identify various geometric features of ultrasonic instrument 10. For example, many ultrasonic instruments provide a surface adapted primarily for cutting and a second surface adapted primarily for cauterizing. The geometric differences between these surfaces are often slight which presents users with a visual challenge when trying to optimize the effectiveness of the instrument. Providing coloration to, for example, the surface of the instrument adapted primarily for cutting will provide users with a clear indicator of the instrument feature that is most desirable in a given circumstance.

Another embodiment of the present invention includes providing coloration to a portion of ultrasonic instrument 10 by anodizing the instrument with an anodizing pen. The anodizing pen may be used to write on the ultrasonic instrument to indicate use, model number, length, shape or any other desirable visual indicia. Using an anodizing pen in combination with an ultrasonic instrument provides a permanent and easily visible marking system suitable for use in medical procedures.

Yet another embodiment of a method in accordance with the present invention for providing coloration to an ultrasonic instrument includes providing an ultrasonic instrument and providing the end effector of the ultrasonic instrument with coloration. This step includes providing any ultrasonic instrument suitable for use in a medical capacity having an end effector. Ultrasonic instruments in accordance with this method may be instruments suitable for clinical, laboratory, and/or surgical use. This method includes providing coloration to distal end effector portion of the ultrasonic instrument by, for example, anodizing, providing permanent inks, using chemical vapor deposition, providing a powder coating, chemical processing, chemical oxidation, and/or using ion bombardment.

One embodiment of the present invention includes providing ultrasonic instrument 10 with coloration to differentiate between multiple instruments. Due to the small variation between many ultrasonic surgical devices such as, for example, between sharp hooks and dissecting hooks, color variations between instruments may be used to identify instruments being used in particular medical procedures. For example, sharp hooks may be provided with a green color covering all or a portion of the device and dissecting hooks may be provided with a red color covering all or a portion of the device. Such coloration may be used in a combination with coloration used to detect early blade deterioration.

Another embodiment of a method in accordance with the present invention for providing different coloration to ultrasonic instruments having different functions includes providing multiple ultrasonic instruments having different functions and providing ultrasonic instruments with different coloration based on functionality differences. This method includes providing more than one ultrasonic instrument having different functions, shapes, costs, and/or configurations. Ultrasonic instruments in accordance with this embodiment may be instruments suitable for clinical, laboratory, and/or surgical use. This embodiment includes providing a different coloration to each of the ultrasonic devices in order to prevent confusion as to the type and/or function of the ultrasonic instrument being used. For example, sharp hook ultrasonic devices may be provided with a green color and dissecting hooks may be provided with a blue color.

A further embodiment of the present invention includes providing coloration to all or a portion of ultrasonic instrument 10 as an indicator of when a disposable instrument or single-use instrument, has been previously used. By providing this coloration it will be more difficult for disposable instruments to be used accidentally in multiple procedures and will protect patients from instrument failures that occur when such instruments are improperly used more than once. Coloration may be applied in, for example, bands, designs, or over the entirety of the device to provide a clear indicator of previous use.

Yet another embodiment of a method in accordance with the present invention for preventing the re-use of disposable ultrasonic instruments includes providing a disposable ultrasonic instrument and providing the ultrasonic instrument with coloration that changes upon use of the instrument. Ultrasonic instruments in accordance with this embodiment may be disposable instruments suitable for clinical, laboratory, and/or surgical use. The method includes providing the ultrasonic instrument with coloration that changes with use to a permanently different color to indicate that a disposable blade has been used. Such pigments include thermochromatic inks, paints, and dyes such as those sold by the H.W. SANDS CORPORATION, Jupiter, Fla. For example, a disposable instrument may be colored orange with a thermochromatic pigment before use where the heat produced during the use of the instrument changes the color of the instrument from orange to red. The thermochromatic pigment is preferably applied to inactive portions of the ultrasonic instrument that will change color as heat disperses throughout the instrument. Such coloration may be applied through any mechanical or manual painting or dying means. The instrument, after use, will retain the red color to indicate that the instrument should not be reused.

Additionally, coloration may be used in re-usable instruments to demonstrate early signs of blade deterioration to users. For example, as ultrasonic instruments begin to deteriorate, their heat dispersion generally increases. One embodiment of the present invention includes providing a re-usable ultrasonic instrument that changes color for example, from green to red, when the instrument begins to overheat. This will provide an early indicator of possible blade damage and will also alert physicians that they may be desiccating surrounding tissue unintentionally due to an overheated instrument. The coloration of ultrasonic instrument 10 may be accomplished through the use of thermochromatic pigments such as those sold by H.W. SANDS CORPORATION, Jupiter, Fla., and includes the use of coloration that, once changed, is permanent, or pigments that return to their initial color as the temperature of the instrument drops. This coloration may be performed by any mechanical or manual painting or dying means.

Another method in accordance with the present invention includes a method of providing coloration to a reusable device to demonstrate when the ultrasonic instrument may be overheating. This embodiment includes providing a re-usable ultrasonic instrument and providing the ultrasonic instrument with coloration that changes upon use of the instrument. Ultrasonic instruments in accordance with this embodiment may be re-usable instruments suitable for clinical, laboratory, and/or surgical use. This embodiment includes providing the ultrasonic instrument with coloration that changes with use to a different color to indicate that the instrument is overheating and poses a potential danger to the patient. Such a color change may be permanent or, upon cooling down, the instrument may return to its previous color.

Each feature disclosed in this specification (including accompanying claims, abstract, and drawings), may be replaced by alternative features having the same, equivalent, or similar purpose, unless expressly stated otherwise. Thus, unless expressly stated otherwise, each feature disclosed is on example only of a generic series of equivalent or similar features.

While preferred embodiments of the present invention have been shown and described herein, it will be obvious to those skilled in the art that such embodiments are provided by way of example only. Numerous variations, changes, and substitutions will be apparent to those skilled in the art without departing from this invention. Accordingly, it is intended that the invention be limited only by the spirit and scope of the appended claims.

1. A condition-indicating ultrasonic surgical device, comprising:
an ultrasonic surgical instrument; and
at least a portion of the instrument having an outer surface including a color layer of a predetermined thickness formed by anodizing or chemical processing;
wherein the thickness of the layer is affected by damage to the instrument resulting in a color change that indicates the presence of damage that would otherwise be imperceptible by visual inspection.

2. The device of claim 1, wherein the instrument is an ultrasonic scalpel.

3. The device of claim 1, wherein the color layer portion includes an end effector portion.

4. The device of claim 1, wherein the color layer portion includes an antinode portion of the instrument.

5. The device of claim 1, wherein the color layer portion includes substantially the entire outer surface of the instrument.

6. The device of claim 1, wherein the instrument is made of a material comprising titanium.

7. The device of claim 6, wherein the color layer is formed by anodization to provide a substantially dark blue color with a thickness of about 586 angstroms.

8. The device of claim 6, wherein the color layer is formed by anodization to provide a substantially light purple color with a thickness of about 1573 angstroms.

9. The device of claim 1, wherein the instrument is made of a material comprising titanium and wherein the color layer is formed by anodization to provide a thickness of at least about 240 angstroms.

10. The device of claim 9, wherein the color layer has a thickness in the range of about 400 angstroms to about 1800 angstroms.

11. The device of claim 10, wherein the color layer has a thickness in the range of about 400 angstroms to about 1100 angstroms.

12. The device of claim 11, wherein the color layer has a thickness in the range of about 500 angstroms to about 900 angstroms.

13. The device of claim 9, wherein the color layer has a thickness of no more than about 926 angstroms.

14. The device of claim 9, wherein the color layer has a thickness at least about 926 angstroms.

15. A method of making a condition-indicating ultrasonic surgical device, comprising the steps of:
providing an ultrasonic surgical instrument; and
forming a color layer having a predetermined thickness on at least a portion of an outer surface of the instrument by anodizing or chemical processing such that the color of the layer is affected by damage to the instrument that would otherwise be imperceptible by visual inspection.

16. The method of claim 15, wherein the thickness of the color layer is affected by damage to the instrument, thereby effecting a change in color at a point of such damage.

17. The method of claim 15, wherein the instrument is an ultrasonic scalpel.

18. The method of claim 15, wherein the color layer portion includes an antinode portion.

19. The method of claim 15, wherein the color layer portion includes an end effector portion.

20. The method of claim 15, wherein the color layer portion includes substantially the entire outer surface of the instrument.

21. The method of claim 15, wherein the instrument is made of a material comprising titanium.

22. The method of claim 21, wherein the color layer is formed by anodization using an applied voltage of about 20V to provide a substantially dark blue color with a thickness of about 586 angstroms.

23. The method of claim 21, wherein the color layer is formed by anodization using an applied voltage of about 65V to provide a substantially light purple color with a thickness of about 1573 angstroms.

24. The method of claim 15, wherein the instrument is made of a material comprising titanium and wherein the color layer is formed by anodization using an applied voltage in the range of about 3V to about 100V.

25. The method of claim 24, using an applied voltage in the range of about 13V to about 75V.

26. The method of claim 24, using an applied voltage in the range of about 13V to about 38V.

27. The method of claim 24, using an applied voltage in the range of about 17V to about 30V.

28. The method of claim 24, using an applied voltage in the range of about 20V to about 75V.

29. The method of claim 24, using an applied voltage in the range of about 3V to about 35V.

30. The method of claim 24, using an applied voltage in the range of about 35V to about 100V.
